(19) United States
(12) Patent Application Publication
(10) Pub. No.: US 2021/0035680 A1
(43) Pub. Date: Feb. 4, 2021

(54) Systems and methods for automating clinical workflow decisions and generating a priority read indicator

(71) Applicant: Hologic, Inc., Marlborough, MA (US)

(72) Inventors: Biao CHEN, Newark, DE (US); Zhenxue JING, Chads Ford, PA (US); Ashwini KSHIRSAGAR, Santa Clara, CA (US); Nikolaos GKANATSIOS, Danbury, CA (US); Haili CHUI, Santa Clara, CA (US)

(21) Appl. No.: 16/944,995

(22) Filed: Jul. 31, 2020

Related U.S. Application Data
(60) Provisional application No. 62/881,156, filed on Jul. 31, 2019.
(60) Provisional application No. 62/941,601, filed on Nov. 27, 2019.

(51) Int. Cl. G16H 40/20; G16H 50/20; G16H 30/40; G16H 50/70; G16H 70/20; G06Q 10/06; G06T 7/00; A61B 6/02; A61B 6/00; A61B 8/08

(52) U.S. Cl. CPC G16H 40/20; G16H 50/20; G16H 30/40; G16H 50/70; G16H 70/20; G06Q 10/06316; G16H 15/00; A61B 6/025; A61B 6/502; A61B 8/0825; A61B 6/5217; A61B 8/5223; G06T 7/0014

(57) ABSTRACT

Examples of the present disclosure describe systems and methods for automating clinical workflow decisions. In aspects, patient data may be collected from multiple data sources, such as patient records, imaging data, etc. The patient data may be processed using an artificial intelligence (AI) component. The output of the AI component may be used by healthcare professionals to inform healthcare decisions for patients. The output of the AI component and additional information relating to the healthcare decisions and healthcare paths may be provided as input to a decision analysis component. The decision analysis component may process the input and output an automated healthcare recommendation that may be used to further inform the healthcare decisions of the healthcare professionals. In some aspects, the output of the decision analysis component may be used to determine a priority or timeline for performing one or more actions relating to patient healthcare.

### BACKGROUND

Modern breast care involves an analysis of various complex factors and data points, such as patient history, healthcare professional experience, imaging modality utilized, etc. The analysis enables healthcare professionals to determine the breast care path that will optimize breast care quality and patient experience. However, such determinations are subjective and, thus, may vary substantially from one healthcare professional to another. As a consequence, some patients may be provided with suboptimal breast care paths, resulting in increased hospital costs and a diminished patient experience.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

Examples of the present disclosure describe systems and methods for automating clinical workflow decisions. In aspects, patient data may be collected from multiple data sources, such as patient records, healthcare professional notes/assessments, imaging data, etc. The patient data may be processed using an artificial intelligence (AI) component. The output of the AI component may be used by healthcare professionals to inform healthcare decisions for one or more patients. The output of the AI component, information relating to the healthcare decisions of the healthcare professionals, and/or supplementary healthcare-related information may be provided as input to a decision analysis component. The decision analysis component may process the input and output an automated healthcare recommendation that may be used to further inform the healthcare decisions of the healthcare professionals. In some aspects, the output of the decision analysis component may be used to determine a priority or timeline for performing one or more actions relating to patient healthcare. For example, the output of the decision analysis component may indicate a priority or importance level for evaluating patient imaging data.

Aspects of the present disclosure provide a system comprising: at least one processor; and memory coupled to the at least one processor, the memory comprising computer executable instructions that, when executed by the at least one processor, performs a method comprising: collecting patient data from one or more data sources; providing the patient data to a first artificial intelligence (AI) algorithm for analyzing features of the patient data; receiving a first output from the first AI algorithm; providing the first output to a second AI algorithm for determining clinical workflow decisions for patient care; receiving a second output from the second AI algorithm, wherein the second output comprises an automated patient care recommendation; and providing the automated patient care recommendation to a healthcare professional.

Aspects of the present disclosure further provide a method comprising: collecting patient data from one or more data sources; providing the patient data to a first artificial intelligence (AI) component for analyzing features of the patient data; receiving a first output from the first AI component; providing the first output to a second AI component for determining clinical workflow decisions for patient care; receiving a second output from the second AI component, wherein the second output comprises an automated patient care recommendation; and providing the automated patient care recommendation to a healthcare professional.

Aspects of the present disclosure further provide a system comprising: at least one processor; and memory coupled to the at least one processor, the memory comprising computer executable instructions that, when executed by the at least one processor, performs a method comprising: collecting image data from one or more data sources; evaluating the image data to identify one or more features; calculating a confidence score based on the one or more features; comparing the confidence score to a threshold value; and when the confidence score exceeds the threshold value, assigning an elevated evaluation priority to the image data.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

Non-limiting and non-exhaustive examples are described with reference to the following figures.

FIG. 1 illustrates an overview of an example system for automating clinical workflow decisions, as described herein.

FIG. 2 is a diagram of an example process flow for automating clinical workflow decisions, as described herein.

FIG. 3 illustrates an overview of an example decision processing system for automating clinical workflow decisions, as described herein.

FIG. 4 illustrates an example method for automating clinical workflow decisions, as described herein

FIG. 5 illustrates an example method for determining image reading priority, as described herein.

FIG. 6A illustrates an example user interface that is associated with the automated clinical workflow decisions described herein.

FIG. 6B illustrates an analytics dialog interface associated with the example user interface of FIG. 6A.

FIG. 7 illustrates one example of a suitable operating environment in which one or more of the present embodiments may be implemented.

### DETAILED DESCRIPTION

Medical imaging has become a widely used tool for identifying and diagnosing abnormalities, such as cancers or other conditions, within the human body. Medical imaging processes such as mammography and tomosynthesis are particularly useful tools for imaging breasts to screen for, or diagnose, cancer or other lesions with the breasts. Tomosynthesis systems are mammography systems that allow high resolution breast imaging based on limited angle tomosynthesis. Tomosynthesis, generally, produces a plurality of X-ray images, each of discrete layers or slices of the breast, through the entire thickness thereof. In contrast to conventional two-dimensional (2D) mammography systems, a tomosynthesis system acquires a series of X-ray projection images, each projection image obtained at a different angular displacement as the X-ray source moves along a path, such as a circular arc, over the breast. In contrast to conventional computed tomography (CT), tomosynthesis is typically based on projection images obtained at limited angular displacements of the X-ray source around the breast. Tomosynthesis reduces or eliminates the problems caused by tissue overlap and structure noise present in 2D mammography imaging.

In modern breast care centers, the images produced using medical imaging are evaluated by various healthcare professionals to determine the optimal breast care path for patients. However, this evaluation can be daunting given the complexities of imaging data and systems, patient information and records, hospital information systems, healthcare professional knowledge and experience, clinical practice guidelines, AI diagnostic systems and output, etc. As a result, the evaluation may produce healthcare decisions that vary substantially from one healthcare professional to another. The variance in healthcare decisions may cause some healthcare professionals to provide suboptimal healthcare paths to some patients. These suboptimal healthcare paths may appreciably diminish the patient experience.

Moreover, medical imaging evaluations typically include a batch reading process, for which the image data for numerous screening subjects (e.g., hundreds or more) are collected. Generally, after the screening subjects have departed the imaging facility, the collected image data is evaluated (“read”) in batches as per the availability of the mammography radiologists. When actionable (or potentially actionable) content is identified in the images evaluated during the batch reading process, the respective screening subjects are “recalled” (e.g., called back to the imaging facility) for follow-up imaging and/or biopsy. Due to scheduling and other conflicts, the time delay between screening (image acquisition) and recall may be several days or weeks. This delay may result in undesirable outcomes in cases of, for example, aggressive cancers. The delay may also cause undue stress and anxiety for screening subjects that are eventually determined to have no abnormalities.

To address such issues with suboptimal healthcare decisions, the present disclosure describe systems and methods for automating clinical workflow decisions to support healthcare professional determinations. In aspects, patient data for one or more patients (or “screening subjects”) may be collected from multiple data sources accessible to a healthcare professional, a medical facility, or a service affiliated therewith. Patient data, as used herein, may refer to information relating to patient name/identifier, patient personal information, medical images, vital signs and other diagnostic information, visit history, prior treatments, previously diagnosed conditions/disorders/diseases, prescribed medications, etc. Examples of data sources include, but are not limited to, patient visit information, patient electronic medical records (EMRs), hospital information systems (HISs), and medical imaging systems. In examples, the patient data collection process may be performed manually, automatically, or some combination thereof.

After collecting the patient data, the patient data may be proved to an AI processing component. The AI processing component may utilize one or more rule sets, algorithms, or models. A model, as used herein, may refer to a predictive or statistical utility or program that may be used to determine a probability distribution over one or more character sequences, classes, objects, result sets or events, and/or to predict a response value from one or more predictors. A model may be based on, or incorporate, one or more rule sets, machine learning, a neural network, or the like. In examples, the AI processing component may process the patient data and provide one or more outputs. Example outputs include, but are not limited to, breast composition/density category scores, computer-aided detection markers (e.g., for calcifications and masses detected in the breast), computed radiometric features, breast cancer risk assessment results, etc. A breast composition/density category score, as used herein, may indicate the proportion of a breast that is composed of fibroglandular tissue. Generally, breasts with high density contain a larger amount of epithelial cells, stromal cells, and collagen, which are a significant factor in the transformation of normal cells to cancer cells. Computer-aided detection markers, as used here, may refer to digital geometric forms (e.g., triangles, circles, squares, etc.) added to (or overlaying) an image. The detection markers may indicate areas of the breast in which lesions or diagnostically interesting objects have been detected using computer-aided detection software and/or machine learning algorithms. Radiometric features, as used herein, may refer to characteristics describing the information content in an image. Such characteristics may include image attributes/values relating to breast density, breast shape, breast volume, image resolution, etc.

In aspects, the outputs and/or patient data may be provided to one or more recipients or recipient devices. Examples of recipient devices include, but are not limited to, image review workstations, medical imaging systems, and technician workstations. Healthcare professionals (and/or persons associated therewith) may use the recipient devices to evaluate the outputs and/or patient data in order to inform one or more healthcare decisions or paths. As one particular example, a set of X-ray images of a patient's breast and the outputs of the AI processing component may be provided to an image review workstation. A physician may evaluate the data provided to the image review workstation to determine an initial or primary breast care path for a patient. A breast care path (or a healthcare path), as used herein, may refer to a plan or strategy for guiding decisions and timings for diagnosis, interventions, treatments, and/or supplemental action at one or more stages of a disease or condition. Generally, a breast care path may represent a strategy for managing a patient population with a specific problem or condition (e.g., a care pathway), or managing an individual patient with a specific problem or condition (e.g., a care plan). As another example, the outputs of the AI processing component may be provided to the imaging system or acquisition room. A technologist may evaluate the data provided to the imaging system/acquisition room to enable technologists to perform diagnostic procedures while a patient is on site.

In aspects, various inputs may be provided to a decision analysis component configured to output a recommended healthcare path. The decision analysis component may utilize one or more rule sets, algorithms, or models, as described above with respect to the AI processing component. Example inputs to the decision analysis component include, but are not limited to, patient data, outputs of the AI processing component, healthcare professional's initial/primary healthcare decisions and diagnostic assessments, and healthcare practice guidelines from clinical professional bodies. The decision analysis component may process the various inputs and provide one or more outputs. Example outputs include, but are not limited to, automated patient healthcare recommendations, assessments of healthcare professional decisions, recommended treatments and procedures, instructions for performing treatments/procedures, diagnostic and intervention reports, automatic appointment scheduling, and evaluation priorities or timelines. In examples, the output of the decision analysis component may be provided (or otherwise made accessible) to one or more healthcare professionals. The output may be used to further inform the healthcare decisions of the healthcare professionals.

In some aspects, the decision analysis component output may comprise (or otherwise indicate) a priority read indicator. The priority read indicator may indicate the evaluation (“reading”) priority for one or more medical images. In examples, the priority read indicator may be determined by identifying aspects of a medical image (such as the features of a potentially actionable lesion), determining a level of confidence for the identified aspects, and comparing the determined level of confidence to a threshold value. Those medical images that meet and/or exceed the threshold may be assigned a “priority” status or value. Alternately, the “priority” status or value may be assigned to the patient corresponding to the medical images. The priority status/value may be used to place an evaluation importance or timeline on the reading of a medical image or the further evaluation of a patient. For example, a medical image having a “high” priority status may be placed in a reading queue above medical images of normal or lower priority statuses. As a result of the “high” priority status of the medical image, a healthcare professional may be immediately (or quickly) notified of the medical image and may evaluate the medical image while the screening subject is still at the screening facility. As another example, a patient having a “high” priority status may immediately undergo further evaluation. For instance, additional medical images of the patient may be collected, a medical specialist may immediately meet with (or be assigned to) the patient, or a medical appointment/procedure may be scheduled. The priority read indicator, thus, improves the detection of abnormalities and decreases the number of patient recalls.

Accordingly, the present disclosure provides a plurality of technical benefits including but not limited to: generating an automatic (or semi-automatic) clinical workflow, automating breast care analysis and risk assessment, generating automated treatment and procedure instructions, generating automated diagnostic and intervention reports, enabling “same-visit” diagnostic procedures to be performed while a patient is still on site, normalizing healthcare decision-making, optimizing healthcare recommendations, determining medical image evaluation priority, and increasing patient experience by decreasing patent visits, patient anxiety, hospital costs, and prolonged treatment.

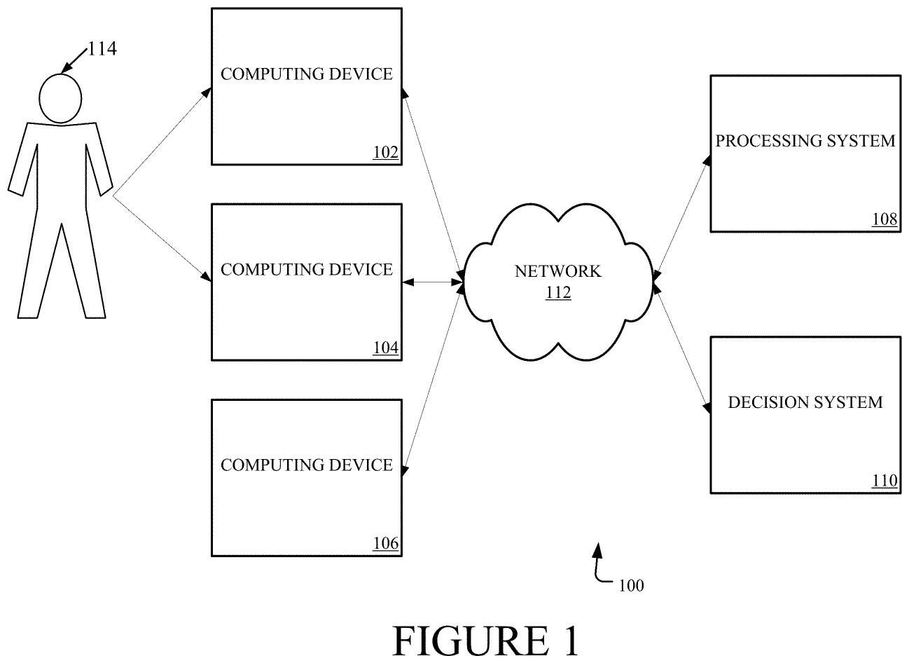

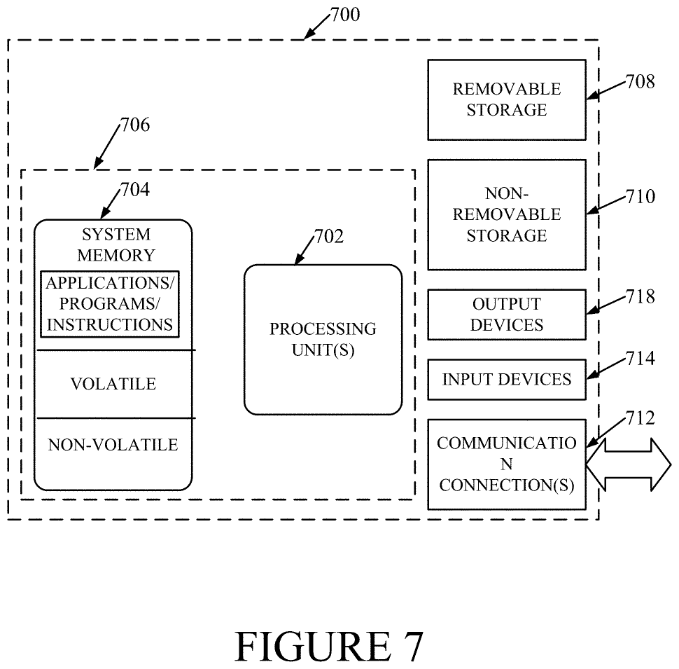

FIG. 1 illustrates an overview of an example system for automating clinical workflow decisions as described herein. Example system 100 as presented is a combination of interdependent components that interact to form an integrated system for automating clinical workflow decisions. Components of the system may be hardware components (e.g., used to execute/run operating system (OS)) or software components (e.g., applications, application programming interfaces (APIs), modules, virtual machines, runtime libraries, etc.) implemented on, and/or executed by, hardware components of the system. In one example, example system 100 may provide an environment for software components to run, obey constraints set for operating, and utilize resources or facilities of the system 100. For instance, software may be run on a processing device such as a personal computer (PC), mobile device (e.g., smart device, mobile phone, tablet, laptop, personal digital assistant (PDA), etc.), and/or any other electronic devices. As an example of a processing device operating environment, refer to the example operating environments depicted in FIG. 7. In other examples, the components of systems disclosed herein may be distributed across multiple devices. For instance, input may be entered on a client device and information may be processed or accessed using other devices in a network, such as one or more server devices.

As one example, the system 100 may comprise computing devices 102, 104, and 106, processing system 108, decision system 110, and network 112. One of skill in the art will appreciate that the scale of systems such as system 100 may vary and may include more or fewer components than those described in FIG. 1. For instance, in some examples, the functionality and components of processing system 108 and decision system 110 may be integrated into a single processing system. Alternately, the functionality and components of processing systems 108 and/or decision system 110 may be distributed across multiple systems and devices.

Computing devices 102, 104, and 106 may be configured to receive patient data for a healthcare patient, such as patient 114. Examples of computing devices 102, 104, and 106 include medical imaging systems/devices (e.g., X-ray, ultrasound, and/or magnetic resonance imaging (MRI) devices), medical workstations (e.g., EMR devices, image review workstations, etc.), mobile medical devices, patient computing device (e.g., wearable devices, mobile phones, etc.), and similar processing systems and devices. Computing devices 102, 104, and 106 may be located in a healthcare facility or an associated facility, on a patient, on a healthcare professional, or the like. In examples, the patient data may be provided to computing devices 102, 104, and 106 using manual or automatic processes. For instance, a healthcare professional may manually enter patient data into a computing device. Alternately, a patient's device may automatically upload patient data to a medical device based on one or more criteria.

Processing system 108 may be configured to process patient data. In aspects, processing system 108 may have access to one or more sources of patient data, such as computing devices 102, 104, and 106, via network 112. At least a portion of the patient data may be provided as input to processing system 108. Processing system 108 may process the input using one or more AI processing techniques. Based on the processed input, processing system 108 may generate one or more outputs, such as breast composition assessment, detection markers, radiometric features, etc. The outputs may be provided (or made accessible) to other components of system 100, such as computing devices 102, 104, and 106. In examples, the outputs may be evaluated by one or more healthcare professionals to determine a healthcare path for a patient. For instance, a physician may use computing device 106 to evaluate X-ray images and/or ultrasound images collected from an imaging system and detection marker results collected from processing system 108. Based on the evaluation, the physician may determine a healthcare decision/plan for a patient.

Decision system 110 may be configured to provide a recommended healthcare path. In aspects, decision system 110 may have access to one or more sources of patient data, outputs from processing system 108, diagnostic assessments and notes, healthcare practice guidelines, and the like. At least a portion of this data may be provided as input to decision system 110. Decision system 110 may process the input using one or more AI processing techniques or models. For example, decision system 110 may implement an artificial neural network, a support vector machine (SVM), a linear reinforcement model, a random decision forest, or a similar machine learning technique. In at least one example, the AI processing techniques performed by decision system 110 may be the same as (or similar to) those performed by processing system 108. In such an example, the functionality of decision system 110 and processing system 108 may be combined into a single processing system or component. Based on the processed input, decision system 110 may generate one or more outputs, such as automated diagnoses, patient care recommendations, assessments of healthcare professional decisions, step-by-step procedure instructions, etc. In aspects, the output(s) may be used to further inform the healthcare decisions of healthcare professionals. For example, a physician may compare a healthcare decision of decision system 110 to the physician's own healthcare decision to determine an optimal healthcare path for a patient.

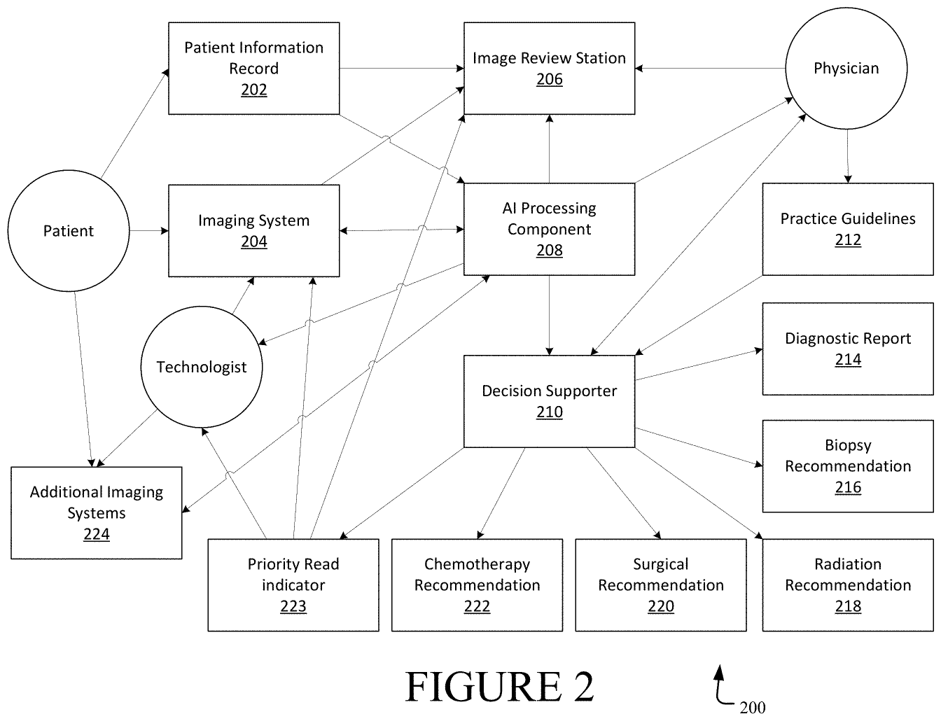

FIG. 2 is a diagram of an example process flow for automating clinical workflow decisions, as described herein. Example process flow 200, as presented, comprises patient information record 202, imaging system 204, image review station 206, AI processing component 208, decision supporter 210, practice guidelines 212, diagnostic report 214, biopsy recommendation 216, radiation recommendation 218, surgical recommendation 220, chemotherapy recommendation 222, priority read indicator 223, and additional imaging system(s) 224. One of skill in the art will appreciate that the scale of systems such as system 200 may vary and may include more or fewer components than those described in FIG. 2.

As illustrated in FIG. 2, patient data may be collected from a patient. In some aspects, the patient data may be collected from the patient during a visit to a healthcare facility. In other examples, the patient data may be provided to the healthcare facility while the patient is not visiting the healthcare facility. For example, the patient data may be uploaded to one or more HIS devices remotely from a patient device. In process flow 200, patient information record 202 may store patient information such as name or identifier, contact information, personal information, diagnostic history, vital signs information, prescribed medications, etc. Imaging system 204 may generate and/or store, for example, X-ray breast images of a patient. Additional imaging system(s) 224 may generate and/or store, for example, ultrasound breast images and/or Mill breast images of a patient.

In aspects, the information recorded in patient information record 202 and the images generated using imaging system 204 and additional imaging system(s) 224 (collectively referred to as “patient data”), may be provided to AI processing component 208. In examples, AI processing component 208 may be configured to assess one or more characteristics of a patient's breast based on breast image data received as input. The assessment may comprise an analysis of imaged breast texture/tissue and an identification of one or more patterns in a breast image. Based on the provided patient data, AI processing component 208 may generate breast assessment data, such as breast composition/density category scores, computer-aided detection markers (e.g., for calcifications and masses detected in the breast), computed radiometric features, and breast cancer risk assessment results. The breast assessment data may be provided to imaging system 204 and/or additional imaging system(s) 224. A technologist may evaluate the breast assessment data provide to imaging system 204 and/or additional imaging system(s) 224 to determine, for example, whether to perform additional imaging for the patient. The breast assessment data and/or patient data may also be provided to image review station 206. A physician may evaluate the information provided to image review station 206, as well as practice guidelines 212, to create diagnostic information and/or healthcare decisions for the patient (collectively referred to as “diagnostic report”).

In aspects, the breast assessment data, patient data, and/or diagnostic report may be provided to decision supporter 210. Based on the provided information and/or practice guidelines 212, decision supporter 210 may automatically generate decision information, such as patient healthcare recommendations, assessments of healthcare professional decisions, recommended imaging procedures, recommended treatments and procedures, instructions for performing treatments/procedures, priorities and/or timelines for treatments/procedures, and diagnostic report 214. Examples of recommended treatments and procedures include biopsy recommendation 216, radiation recommendation 218, surgical recommendation 220, and chemotherapy recommendation 222. Examples of treatment and procedure priorities/timelines include priority read indicator 223. Priority read indicator 223 may comprise or represent a status, value, or date/time for evaluating a medical image. In some aspects, the decision information may be made accessible to one or more healthcare professionals (or to computing devices associated therewith). For example, process flow 200 depicts the decision information being provide to the physician that created the diagnostic report. As another more specific example, process flow 200 depicts priority read indicator 223 being provided to a technologist, imaging system 204, and image review station 206.

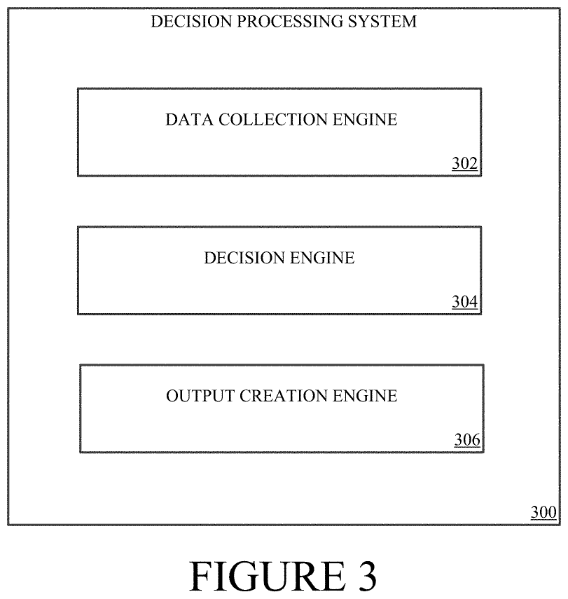

FIG. 3 illustrates an overview of an example decision processing system 300 for automating clinical workflow decisions, as described herein. The automated clinical workflow techniques implemented by input decision system 300 may comprise the automated clinical workflow techniques and data described in the system of FIG. 1. In some examples, one or more components (or the functionality thereof) of input decision system 300 may be distributed across multiple devices and/or systems. In other examples, a single device (comprising at least a processor and/or memory) may comprise the components of input decision system 300.

With respect to FIG. 3, input decision system 300 may comprise data collection engine 302, decision engine 304, and output creation engine 306. Data collection engine 302 may be configured to access a set of data. In aspects, data collection engine 302 may have access to information relating to one or more patients. The information may include patient data (e.g., patient identification, patient medical images, patient diagnostic information, etc.), breast composition assessment, detection markers, radiometric features, diagnostic assessments and notes, healthcare practice guidelines, and the like. In some aspects, at least a portion of the information may be test data or training data. The test/training data may include labeled data and images used to train one or more AI models or algorithms.

Decision engine 304 may be configured to process the received information. In aspects, the received information may be provided to decision engine 304. Decision engine 304 may apply one or more AI processing algorithm or models to the received information. For example, decision engine 304 may apply an AI-based fusion algorithm to the received information. The AI processing algorithms/models may evaluate the received information to determine correlations between the received information and training data used to train the AI processing algorithms/models. Based on the evaluation, decision engine 304 may identify or determine an optimal healthcare path or recommendation for one or more patients associated with the patient data. In some aspects, decision engine 304 may further identify and provide an image reading priority. For instance, decision engine 304 may assign a “priority” status to an image in the received information.

Output creation engine 306 may be configured to create one or more outputs for received information. In aspects, output creation engine 306 may use the identifications or determinations of decision engine 304 to create one or more outputs. As one example, output creation engine 306 may recommend the use of one or more additional imaging modalities, such as contrast enhanced MRI, advanced ultrasound imaging (e.g., shear waving imaging, contrast imaging, 3D imaging, etc.), and positron emission tomography (PET) imaging. As another example, output creation engine 306 may generate a comprehensive report comprising diagnostic information and recommendations for biopsy procedures, chemotherapy, surgical intervention, or radiation therapy. The recommendation may include detailed procedural instruction and correlations between data points and medical images. As a specific example, for biopsy procedures, output creation engine 306 may provide step by step biopsy instructions with correlated biopsy images and previous diagnostic images from X-ray, ultrasound, and MRI imaging systems.

Having described various systems that may be employed by the aspects disclosed herein, this disclosure will now describe one or more methods that may be performed by various aspects of the disclosure. In aspects, methods 400 and 500 may be executed by an example system, such as system 100 of FIG. 1 or decision processing system 300 of FIG. 3. In examples, methods 400 and 500 may be executed on a device comprising at least one processor configured to store and execute operations, programs, or instructions. However, methods 400 and 500 are not limited to such examples. In other examples, methods 400 and 500 may be performed on an application or service for automating clinical workflow decisions. In at least one example, methods 400 and 500 may be executed (e.g., computer-implemented operations) by one or more components of a distributed network, such as a web service/distributed network service (e.g., cloud service).

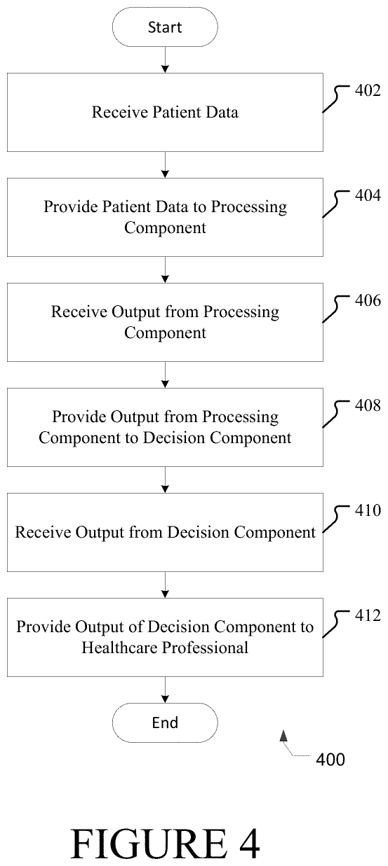

FIG. 4 illustrates an example method 400 for automating clinical workflow decisions as described herein. Example method 400 begins at operation 402, where patient data is collected from one or more data sources. In aspects, a data collection component, such as data collection engine 202, may collect patient data from one or more data sources. Example data sources include patient visit information, patient EMRs, medical facility HIS records, and medical imaging systems. For instance, during a patient visit to a healthcare facility, a patient information record stored by (or accessible to) the healthcare facility may be used to collect or access a patient's personal information, such as patient age, diagnostic history, lifestyle information, etc. During the patient visit, an imaging system may be used to generate one or more images of the patient's breast. For example, an X-ray imaging system may acquire or generate 2D images and/or tomosynthesis images of the patient's breasts. In another example, an ultrasound system may acquire one or more images of the patient's breasts. The images may be combined (or otherwise correlated) with the personal information and/or stored in one or more medical records or medical systems of the healthcare facility. In another instance the images collected may be provided directly to the processing component as described below.

In aspects, the data collection process may be initiated manually and/or automatically. For example, a healthcare professional may manually initiate the data collection process by soliciting patient information from the patient and entering the solicited patient information into a patient information record. Alternately, the data collection process may be initiated automatically upon the satisfaction of one or more criteria. Example criteria may include, a patient check-in event, a scheduled appointment, entering diagnostic information or a patient healthcare path into the HIS, or evaluating digital mammography images via an image review workstation. For instance, in response to detecting a patient scheduled appointment at a healthcare facility, an electronic system/service of the healthcare facility may automatically collect patient information from one or more of the patient's medical records. The collected data may be aggregated into an active working file or patient case file for the patient visit.

At operation 404, the patient data is provided to a processing component. In aspects, one or more portions of the patient data may be provided to a processing component, such as AI processing component 208. The processing component may be, comprise, or have access to one or more rule sets, algorithms, or predictive models. The processing component may use a set of AI algorithms to process the information and create a group of outputs. For instance, continuing from the above example, the combined data (e.g., the personal information and the images of the patient) may be provided as input to an AI system accessible to the healthcare facility. The AI system may be implemented on a single device (such as a single workstation of the healthcare facility) or provided as a distributed service/system to multiple device across multiple devices in a distributed computing environment. The AI system may be configured to perform breast assessment using a machine-learning algorithm that analyzes each patient's breast attributes (such as patterns, textures, etc.). The AI system may be implemented on a single device (such as a single workstation of the healthcare facility) or provided as a distributed service/system to multiple device across multiple devices in a distributed computing environment. By applying the machine-learning algorithm to the combined data, the AI system may identify one or more aspects of the images that indicate the imaged breast is heterogeneously dense. This density classification may be based on, for example, the American College of Radiology (ACR) Breast Imaging Reporting and Data System (BI-RADS) mammographic density (MD) assessment categories. The AI system may further add detection markers to the images to indicate one or more calcifications or masses detected in the images.

At operation 406, output is received from the processing component. In aspects, the processing component may create one or more outputs from the patient data. Example outputs include breast composition category scores, breast density assessments, computer-aided detection markers, computed radiometric features, breast cancer risk assessment results, etc. At least a portion of the output may be provided to one or more healthcare professionals and/or healthcare systems/devices. For instance, continuing from the above example, the AI system may output the density classification of the imaged breast (e.g., heterogeneously dense) and/or the corresponding image data (e.g., the original images, the image updates with detection markers, and/or calcification or mass data, etc.). The AI system output may be provided to one or more computing devices (e.g., workstations, mobile devices, etc.) of the patient's radiologist and/or a medical imaging technologist. Based on the radiologist's evaluation of the AI system output, the radiologist may recommend performing ultrasound imaging of the patient's breast. In response to the recommendation, the medical imaging technologist may perform recommended diagnostic procedures (e.g., magnification/contact diagnostic view imaging) and/or supplemental screening procedures (e.g., ultrasound imaging) while patients are still on site (e.g., during the current patient visit). Performing these procedures while the patients are still on site may avoid additional medical facility visits and reduce medical costs associated with rescheduling appointments.

At operation 408, output of the processing component is provided to a decision component. In aspects, the output of the processing component, healthcare professional recommendations, image data, supplemental data from diagnostic/screening procedures, and other patient-related information may be provided to a decision component, such as decision engine 304. The decision component may be, comprise, or have access to one or more rule sets, algorithms, or predictive models. The decision component may use one or more AI algorithms to process the information and create a group of outputs. For instance, continuing from the above example, patient data, AI system output, X-ray image data, ultrasound image data (recommended by the radiologist), the radiologist's recommendation data, and practice guidelines from one or more clinical professional bodies (e.g., American College of Radiology (ACR), National Comprehensive Cancer Network (NCCN), etc.) may be provided as input to an AI-based fusion algorithm. The AI-based fusion algorithm may be configured to provide an optimal healthcare path or recommendation for one or more patients. Based on the provided input, the AI-based fusion algorithm may determine that a surgical intervention is the optimal care plan for the patient.

At operation 410, output is received from the decision component. In aspects, the decision component may create one or more outputs from the received input. Example outputs include automated patient healthcare recommendations, assessments of healthcare professional decisions, recommended treatments and procedures, instructions for performing treatments/procedures, diagnostic and intervention reports, and automatic appointment scheduling. For instance, continuing from the above example, based on the input provided to the AI-based fusion algorithm, the AI-based fusion algorithm may output a comprehensive report comprising diagnostic information for the patient and a recommendation for surgical intervention for the patient. The recommendation for surgical intervention may be accompanied by specific guidelines for performing the recommended surgical procedure. The instructions may comprise surgical images, step-by-step surgical instructions, computer-aided detection markers, recommended medications, recovery procedures, and the like.

At operation 412, an automated patient healthcare recommendation is provided to a healthcare professional. In aspects, the output from the decision component (or portions thereof) may be provided to one or more targets. Example targets include healthcare professional devices, medical facility devices, patient devices, data archives, one or more processing systems, or the like. The targets may assess the automated patient healthcare recommendation to inform or evaluate the target's own patient healthcare recommendation. For instance, continuing from the above example, the comprehensive report and recommendation for surgical intervention may be provided to one or more computing devices of patient's radiologist. The comprehensive report may indicate that 93% of radiologists have recommended surgical intervention for patients having similar patient data to the patient and similar AI system outputs to the patient's outputs. Based on the comprehensive report and the recommendation, the radiologist may create or approve a recommendation for surgical intervention. In at least one example, the radiologist may modify a previous healthcare recommendation created by the radiologist to be consistent with the recommendation provided by the decision component.

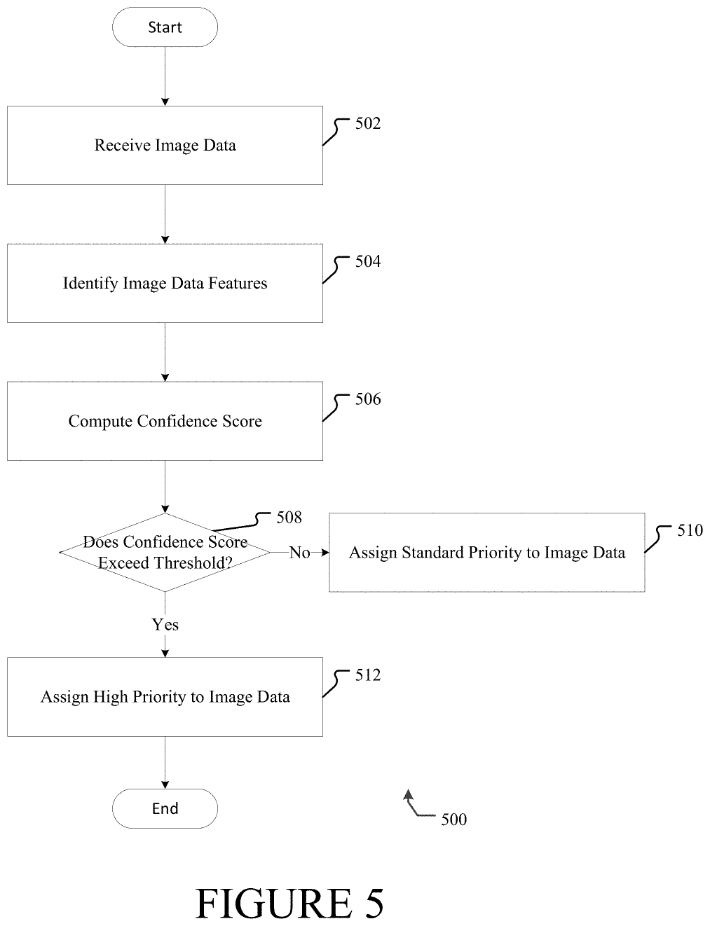

FIG. 5 illustrates an example method 500 for determining image reading priority as described herein. In aspects, example method 500 may be performed (entirely or in part) on an imaging system or device, such as imaging system 204. The imaging system 204 can include an x-ray imaging system or an ultrasound system (as well as other imaging systems). In one example, the X-ray imaging system may include a workstation computer providing operating instructions to the X-ray acquisition system. Example method 500 begins at operation 502, where image data is collected from one or more data sources. In aspects, a data collection component, such as data collection engine 302, may collect image data from one or more data sources. Example data sources include patient EMRs, healthcare facility HIS records, and medical imaging systems. For instance, during a visit to an imaging facility, imaging system 204 may be used to generate one or more 2D and/or tomosynthesis X-ray images of a patient's breasts. The X-ray images may be combined (or otherwise correlated) with personal information of a patient and/or stored in one or more medical records or medical systems of a healthcare facility.

At operation 504, features of the image data may be identified. In aspects, the image data (or portions thereof) may be provided to an input processing component, such as AI processing component 208 and/or decision supporter 210. In at least one example, the input processing component may be incorporated into the imaging system or device on which example method 500 is performed. The image data may be provided to the input processing component as the image data is being collected (e.g., in real-time), immediately after the image data has being collected, or at any other time after the image data has being collected. The input processing component may be, comprise, or have access to one or more rule sets, algorithms, or predictive models. The input processing component may evaluate the image data to identify one or more features of the image data. Image features may include, but are not limited to, shape edges or boundaries, interest points, and blobs. Identifying the features may include the use of feature detection and/or feature extraction techniques. Feature values may be calculated for and/or assigned to the respective features using one or more featurization techniques, such as ML processing, normalization operations, binning operations, and/or vectorization operations. The feature values may be a numerical representation of the feature, a value paired to the feature in the merged data, an indication of one or more condition states for the feature, or the like.

At operation 506, a confidence score may be computed for the image features. In aspects, the input processing component (or a component associated therewith) may use the feature values calculated for an identified image feature to generate a confidence score. The confidence score may represent a probability that a specific feature matches a predefined feature or feature category/classification. Generating the confidence score may include comparing the features and/or feature values of the image data to a set of labeled, known, or predefined features and/or feature values. For example, for a received image, four points of interest may be identified and assigned respective sets of feature values. The respective sets of feature values may each be compared to stored feature data from known images. The stored feature data may comprise various feature values and may be labeled to classify the feature or image. For instance, a set of feature data may be listed for various breast abnormalities and/or mammogram findings. The confidence score may be generated based on matches or similarities between the feature values for the received image and the stored feature values. In aspects, the confidence score may be a numerical value, a non-numerical (or partially numeric) value, or a label. For example, a confidence value may be represented by a numeric value on a scale from 1 to 10, with “1” representing a low confidence of a match and “10” representing a high confidence of a match. In such an example, a higher confidence value may indicate a large number (or percentage) of matches or similarities between the feature values for the received image and the stored feature values.

At decision operation 508, the confidence score may be compared to a threshold value. In aspects, the input processing component (or a component associated therewith) may compare the confidence score to a configurable confidence threshold value. The confidence threshold value may represent the level of confidence that must be met or exceeded before an image (or image data) is assigned a priority reading status. The confidence threshold value may be selected based on a desired balance between positive screening cases (e.g., confirmed cancer cases) and negative screening cases (e.g., cases where no cancer was found). For instance, in a particular example, a selected confidence threshold value may result in the identification of a set of 1,000 cases in which 70% of the cases are positive screening cases, 20% of the cases indicate non-cancerous abnormalities, and 10% of the cases are negative screening cases. Each of the positive screening cases may be assigned a priority reading status. By increasing the selected confidence threshold value, a reduced set of cases may be selected. For instance, a set of 750 cases may be identified, in which 80% of the cases are positive screening cases, 15% of the cases indicate non-cancerous abnormalities, and 5% of the cases are negative screening cases. Alternately, by decreasing the selected threshold value, an increased set of cases may be selected. For instance, a set of 1,250 cases may be identified, in which 60% of the cases are positive screening cases, 25% of the cases indicate non-cancerous abnormalities, and 15% of the cases are negative screening cases.

In some examples, the confidence threshold value may be determined and configured manually. For instance, a user may select or modify a confidence threshold value using a user interface of the input processing component. The selection of a confidence threshold value may be based on various factors. For instance, a confidence threshold value may be selected for at least a portion of the imaging systems associated with a particular medical facility based on whether a sufficient amount of radiologists are associated with the medical facility, or how quickly radiologists are able to review cases with a priority reading status. In other examples, the confidence threshold value may be determined automatically and/or dynamically by the input processing component. For instance, feedback or output relating to a suggested healthcare path, an image reading priority, etc. from one or more entities or components of system 200 may be accessible to the input processing component. The feedback/output may include accuracy ratings or comments from technologists, physicians, or radiologists. The feedback/output may additionally include treatment reports, patient notes, etc. Based on the feedback/output, the input processing component may modify the threshold value to increase or decrease the number of positive and/or negative screening cases identified.

In aspects, if the confidence score is determined to be below the confidence threshold value, flow proceeds to operation 510. At operation 510, the received image data may be assigned a standard level of priority (e.g., standard priority level, low priority level, or no priority level). A standard level of priority may be indicative that the received image data is to be evaluated per the normal availability and/or workload of relevant healthcare professionals. For example, when image data is assigned a standard level of priority, the image data may be added to an image reading queue. The position of the image data in the queue (e.g., the order in which the image data was added to the queue) may dictate the evaluation order of the image data. As a particular example, in a first-in first-out (FIFO) queue, any standard priority data items added to the queue prior to the received image data will be evaluated before the received image data. In such an example, the image data may not be evaluated while the screening subject is still on site at the screening facility.

If, however, the confidence score is determined to meet or exceed the confidence threshold value, flow proceeds to operation 512. At operation 512, the received image data may be assigned a high level of priority. Assigning the high level of priority may comprise, for example, adding one or more indicators to image data and/or metadata, such as the Digital Imaging and Communications in Medicine (DICOM) header for the image data. Example indicators include may include a label (e.g., “High Priority,” “Priority,” etc.), a numerical value, highlighting, arrows or pointers, font or style modifications, date/time values, etc. In aspects, a high level of priority may be indicative that the received image data is to receive prioritized evaluation. As one example, when image data is assigned a high level of priority, the image data may be added to an image reading queue. Based on the high level of priority, the image data may be evaluated before other data items in the queue having lower priority levels and/or later queue entry times/dates. As another example, the priority indicator for image data assigned a high level of priority may be presented to one or more healthcare professionals. For instance, upon assignment of a high level of priority to image data, the priority indicator and/or the image data may be presented to a technologist using a user interface of an X-ray imaging system or device. In at least one instance, the priority indicator and/or the image may be presented to the technologist while the technologist is collecting image data (e.g., in real-time). As yet another example, when image data is assigned a high level of priority, the image data (or an indication thereof) may be transmitted to one or more destinations. For instance, a radiologist may receive a message (e.g., email, text, voice call, etc.) regarding the prior assignment of the image data. The message may comprise information such as the current state or location of the patient, the reading priority for the image data, current and/or past medical records for the patient, etc. As a specific example, image data comprising a priority read indicator may be sent to a radiologist's image review workstation along with an indication that the patient is currently in the medical facility and awaiting a reading of the image data. Alternately, the image data may be sent to a software application or service that is used to manage radiologist workflow. The software application/service may be configured to create and/or assign a worklist of cases that require immediate evaluation. In such examples, the high priority reading indication may enable follow-up imaging and other actions to be performed while the screening subject is still on site at the screening facility.

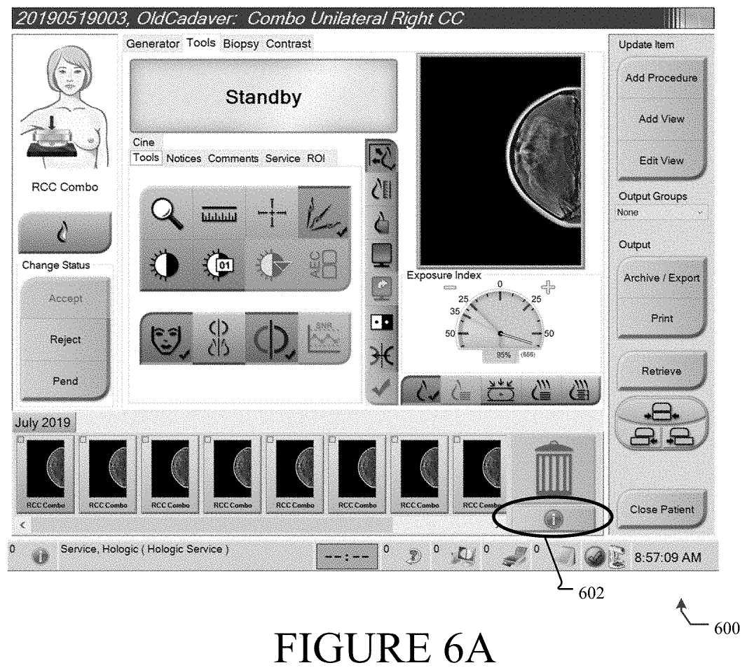

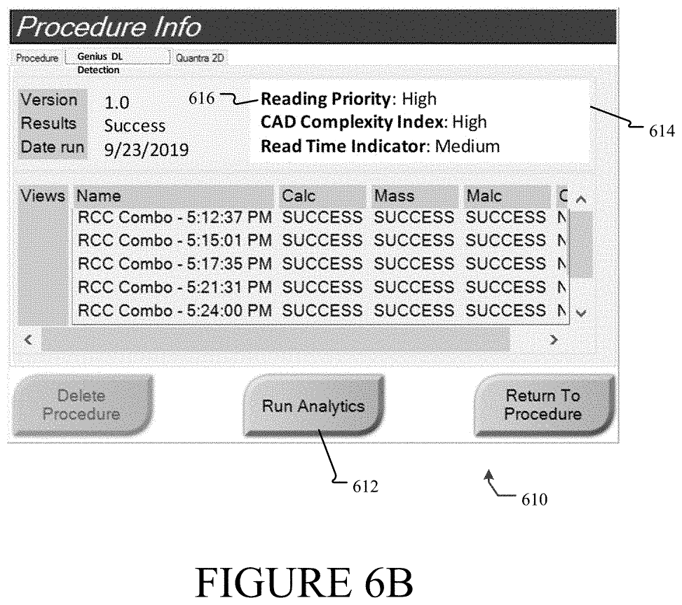

FIG. 6A illustrates an example user interface 600 that is associated with the automated clinical workflow decisions described herein. In examples, user interface 600 represents software a technologist uses on a mammography acquisition workstation. The software may be used to collect images during a breast screening exam from an X-ray imaging system, such as Imaging system 204, and/or to review collected images during a breast screening exam. User interface 600 comprises button 602, which activates an “Analytics” dialog when selected. The “Analytics” dialog may display procedure information relating to one or more patients. In at least one example, selecting button 602 may cause the display of a list of patients that have been marked with a high priority reading. The list may include at least patient name and procedure completion time. The list may be arranged chronologically such that the oldest entry in the list is presented first or at the top of the list and the most recent entry in the list is presented last or at the bottom of the list. In some examples, the appearance of button 602 and/or other elements of user interface 600 may be modified to indicate a reading priority. For instance, when image data has been assigned a reading priority, button 602 may be highlighted, animated, enlarged, encircled, etc. Alternately, a color, a font, a style, etc. of button 602 may be modified.

FIG. 6B illustrates Analytics dialog 610, which is displayed when button 602 of FIG. 6A is selected. Analytics dialog 610 comprises button 612, analysis result section 614, and reading priority indication 616. In aspects, when button 612 is selected, image evaluation software is launched and one or more collected images are analyzed using the techniques described in FIG. 3 and FIG. 4. As a result of the analysis, analysis result section 614 is at least partially populated with data, such as reading priority indication 616. In FIG. 6B, reading priority indication 616 indicates that the reading priority for the analyzed image(s) is “High.” Based on the “High” reading priority, a technologist may request a screening subject to remain on site while a radiologist reviews the collected image(s). This immediate review (e.g., while the screening subject is on site) by the radiologist may mitigate or eliminate the need to recall the screening subject for a follow-up appointment.

FIG. 7 illustrates an exemplary suitable operating environment for the automating clinical workflow decision techniques described in FIG. 1. In its most basic configuration, operating environment 700 typically includes at least one processing unit 702 and memory 704. Depending on the exact configuration and type of computing device, memory 704 (storing, instructions to perform the techniques disclosed herein) may be volatile (such as RAM), non-volatile (such as ROM, flash memory, etc.), or some combination of the two. This most basic configuration is illustrated in FIG. 7 by dashed line 706. Further, environment 700 may also include storage devices (removable, 708, and/or non-removable, 710) including, but not limited to, magnetic or optical disks or tape. Similarly, environment 700 may also have input device(s) 714 such as keyboard, mouse, pen, voice input, etc. and/or output device(s) 716 such as a display, speakers, printer, etc. Also included in the environment may be one or more communication connections 712, such as LAN, WAN, point to point, etc. In embodiments, the connections may be operable to facility point-to-point communications, connection-oriented communications, connectionless communications, etc.

Operating environment 700 typically includes at least some form of computer readable media. Computer readable media can be any available media that can be accessed by processing unit 702 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory medium which can be used to store the desired information. Computer storage media does not include communication media.

Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, microwave, and other wireless media. Combinations of the any of the above should also be included within the scope of computer readable media.

The operating environment 700 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

The embodiments described herein may be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices may be employed to perform the functionality disclosed herein without departing from the scope of the disclosure.

This disclosure describes some embodiments of the present technology with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art.

Although specific embodiments are described herein, the scope of the technology is not limited to those specific embodiments. One skilled in the art will recognize other embodiments or improvements that are within the scope and spirit of the present technology. Therefore, the specific structure, acts, or media are disclosed only as illustrative embodiments. The scope of the technology is defined by the following claims and any equivalents therein.

What is claimed is:

1. A system associated with an imaging device for imaging a breast of a patient, the system comprising:
a display;
at least one processor; and
memory coupled to the at least one processor, the memory comprising computer executable instructions that, when executed by the at least one processor, performs a method comprising:
receiving image data from the imaging device;
providing the image data to an artificial intelligence (AI) component in real-time;
evaluating, by the artificial intelligence (AI) component, the image data to identify one or more features indicative of abnormalities in the breast;
calculating a confidence score based on the one or more features;
comparing the confidence score to a threshold value;
when the confidence score exceeds the threshold value, assigning an elevated evaluation priority to a patient case of the patient; and
displaying a priority read indicator on the display.

2. The system of claim 1, wherein the image data comprises at least one of a 2D X-ray image, a plurality of tomosynthesis X-ray images of a patient breast, or an ultrasound image.

3. The system of claim 1, further comprising receiving patient data from one or more data sources comprise at least one of: patient visit information, patient electronic medical records (EMRs), hospital information system (HIS) records, or medical imaging systems.

4. The system of claim 1, wherein the patient data is evaluated by the AI component and used in part to calculate the confidence score.

5. The system of claim 1, wherein the one or more features comprise at least one of shape edges, shape boundaries, interest points, or blobs.

6. The system of claim 1, wherein identifying the one or more features comprises calculating feature vectors using at least one of machine learning (ML) processing, normalization operations, binning operations, or vectorization operations.

7. The system of claim 1, wherein the confidence score represents a probability that a specific feature of the one or more features matches a predefined feature.

8. The system of claim 1, wherein calculating the confidence score comprises comparing the one or more features to a set of labeled features of one or more previously classified images.

9. The system of claim 1, wherein the threshold value is selected based on a desired balance between positive screening cases and negative screening cases.

10. The system of claim 1, wherein the threshold value is selected based on at least one of whether a sufficient number of radiologists are associated with a medical facility or an amount of time until the radiologists are able to review cases with an elevated evaluation priority.

11. The system of claim 1, wherein the threshold value is configured dynamically by the system after collecting the image data.

12. The system of claim 11, wherein the threshold value is configured dynamically based on information relating to a suggested healthcare path.

13. The system of claim 13, wherein:
when the patient case is assigned the elevated evaluation priority, the patient case is added to a priority queue having a position higher than another case without the elevated priority.

14. The system of claim 1, wherein assigning the elevated evaluation priority to the patient case comprises adding one or more priority indicators to metadata associated with the image data.

15. The system of claim 1, wherein assigning the elevated evaluation priority to the patient case comprises storing the elevated evaluation priority with the patient case.

16. The system of claim 15, wherein the display is associated with a workstation that is in the same room as the imaging device, the workstation being configured to:
collect the image data;
assign the elevated evaluation priority to the patient case;
store the elevated evaluation priority with the patient case; and
present the priority read indicator on the display based on the elevated evaluation priority.

17. A method comprising:
receiving image data from one or more data sources, the image data includes one or more images of a breast of a patient;
evaluating the image data to identify one or more features, wherein the one or more features correspond to at least one of shape edges or interest points of the breast of the patient;
calculating a confidence score based on the one or more features, wherein calculating the confidence score comprises matching the one or more features to features of labeled or known images;
comparing the confidence score to a threshold value; and
when the confidence score exceeds the threshold value, assigning an elevated evaluation priority to the image data.

18. The method of claim 17, wherein the one or more data sources further comprise patient data.

19. The method of claim 17, further comprising:
in response to assigning the elevated evaluation priority to the image data, providing the image data to a workflow service, wherein the workflow service is configured to manage a worklist of cases.

20. The method of claim 19, further comprising displaying the worklist of cases to a radiologist, wherein the worklist service displays the image data having the elevated evaluation priority higher on the worklist of cases than another set of image data.
